(19) United States
(12) Patent Application Publication
(10) Pub. No.: US 2025/0170073 A1
(43) Pub. Date: May 29, 2025

(54) SYNERGISTIC COMPOSITIONS FOR IMPROVING MINERAL BIOACCESSIBILITY

(71) Applicants: N.V. NUTRICIA, Zoetermeer (NL); NUTRICIA EARLY LIFE NUTRITION (SHANGHAI) CO., LTD., Shanghai (CN)

(72) Inventors: Ingrid Brunhilde RENES, Utrecht (NL); Gabriël THOMASSEN, Utrecht (NL); Jan KNOL, Utrecht (NL); Thomas LUDWIG, Utrecht (NL); Haofang JIN, Shanghai (CN)

(73) Assignees: N.V. NUTRICIA, Zoetermeer (NL); NUTRICIA EARLY LIFE NUTRITION (SHANGHAI) CO., LTD., Shanghai (CN)

(21) Appl. No.: 19/038,723

(22) Filed: Jan. 28, 2025

Related U.S. Application Data
(63) Continuation of application No. PCT/CN2023/110133, filed on Jul. 31, 2023.

(30) Foreign Application Priority Data
Aug. 2, 2022 (WO) PCT/CN2022/109593

(51) Int. Cl. A61K 31/047; A61K 31/231; A61K 31/702; A61K 33/06; A61K 33/26; A61K 33/30; A61K 38/17; A61P 3/02

(52) U.S. Cl. CPC A61K 31/047; A61K 31/231; A61K 31/702; A61K 33/06; A61K 33/26; A61K 33/30; A61K 38/1709; A61P 3/02

(57) ABSTRACT

The present invention lies on the field of synergistic compositions for improving mineral bioaccessibility, in particular iron and/or calcium bioaccessibility. The synergistic compositions comprise lutein, casein phosphopeptide or 1,3-dioleoyl-2-palmitoylglycerol, preferably in combination with a non-digestible carbohydrate. The invention further relates to the use of the compositions in preventing and/or treating mineral deficiency and associated conditions, preferably anaemia.

### FIELD OF THE INVENTION

The present invention lies on the field of synergistic compositions for improving mineral bioaccessibility. Particularly, the present invention concerns compositions for use in preventing and/or treating mineral deficiency, preferably anaemia.

### BACKGROUND OF THE INVENTION

Mineral deficiencies affect billions of individuals of all ages worldwide. Even though mineral deficiency may be linked to disorders affecting the digestive tract's ability to adequately absorb minerals, the most common cause of mineral deficiencies is insufficient nutritional intake. Naturally, this is a relevant public health issue in developing countries, but it has increasingly become a health concern among populational groups with (voluntary) dietary restrictions, such as vegans and vegetarians.

Globally, it is estimated that at least 50% of anaemia cases are linked to iron deficiency. In addition to anaemia, suboptimal levels of iron can increase the risk of maternal and perinatal mortality, lead to early cognitive impairment in aging subjects, and decrease immunity, fitness and work productivity in adults.

Calcium deficiency can reduce bone strength and lead to osteoporosis, which is characterized by fragile bones and an increased risk of falling. Calcium deficiency can also cause rachitis in children leading to abnormal cartilage growth and irreversible changes in the skeletal structure. An increasing body of evidence has been relating optimal calcium levels to a reduced risk of cancer, especially colon and rectum cancer.

Although less common than iron and calcium deficiencies, zinc deficiency can also cause relevant conditions, such as growth retardation in infants, loss of appetite and impaired immune function, hair loss, diarrhea, delayed sexual maturation, impotence, hypogonadism in males, and eye and skin lesions. Weight loss and mental lethargy can also occur.

Fortified foods and supplements have been long used against mineral deficiencies. In particular, iron fortification programs have been credited with improving the iron status of millions of women, infants, and children. However, large amounts of supplemental iron might decrease zinc absorption. On the other hand, high zinc intakes can inhibit copper absorption, sometimes producing copper deficiency and associated anaemia. Therefore, a nutritional approach to improve bioaccessibility of minerals is a desired alternative to mineral supplementation.

WO2013057072A1 relates to a composition comprising at least one N-acetylated oligosaccharide, at least one sialylated oligosaccharide and at least one of fructooligosaccharides (FOS) and/or galactooligosaccharides (GOS), for use in the promotion of magnesium absorption and/or magnesium retention in infants.

WO2015000694A1 relates to a composition comprising lactoferrin-osteopontin-iron complexes for use in the treatment or prevention of iron deficiency.

CN111616232A relates to a milk powder for promoting bone growth in infants. The milk powder comprises, in parts by weight: 3000-4000 parts of raw cow milk or raw goat milk, 100-200 parts of skim milk powder, 100-200 parts of desalted whey powder, 30-80 parts of composite vegetable oil or 1,3-dioleic acid-2-palmitic acid triglyceride, 50-150 parts of solid corn syrup, 50-150 parts of whey protein powder, 30-100 parts of crystalline fructose, 20-50 parts of fructo-oligosaccharide, 10-30 parts of anhydrous cream, 5-20 parts of galacto-oligosaccharide, 1-3 parts of compound vitamin, 1-3 parts of compound mineral and 2.2-9 parts of a calcium promoting component. The calcium-promoting ingredient is selected from casein phosphopeptide, hydrolysed egg yolk powder, and vitamin K2 powder.

JP2008184459 relates to a composition comprising calcium and a carotenoid, preferably cryptoxanthin or derivative thereof, for increasing intake of calcium from the small intestine.

Miquel et al. (“Effects and future trends of casein phosphopeptides on zinc bioavailability”. Trends in Food Science & Technology. Volume 18, Issue 3, March 2007, Pages 139-143) discusses the usefulness of the effects of casein phosphopeptides on zinc bioavailability. Similarly, Hansen et al (“Casein phosphopeptides improve zinc and calcium absorption from rice-based but not from whole-grain infant cereal”. J. Pediatr. Gastroenterol. Nutr. 1997 January;24(1):56-62) describes that casein phosphopeptides addition to infant foods provided to adults improved calcium and zinc absorption from rice-based cereal.

### SUMMARY OF THE INVENTION

Using a simulated digestion model, the inventors have observed that mineral bioaccessibility is significantly increased in the presence of lutein. In particular, the inventors have surprisingly observed that the combination of lutein with calcium phosphopeptide or 1,3-dioleoyl-2-palmitoylglycerol, preferably in the presence of the fermentation products of non-digestible carbohydrates, can synergistically increase mineral bioaccessibility. A novel nutraceutical approach to prevent and/or treat mineral deficiencies, particularly iron, zinc, magnesium and calcium deficiencies and associated conditions, is therefore provided.

Accordingly, in a first aspect, the present invention relates to the non-therapeutic use of a composition comprising lutein for increasing mineral bioaccessibility in a human subject, wherein the composition further comprises at least one of:
a. calcium phosphopeptide, or
b. 1,3-dioleoyl-2-palmitoylglycerol.

In a second aspect, the invention relates to a composition comprising lutein for use in preventing or treating mineral deficiency and/or a condition associated therewith in a human subject, wherein the composition further comprises at least one of:
a. calcium phosphopeptide, or
b. 1,3-dioleoyl-2-palmitoylglycerol.

A further aspect relates to synergistic compositions according to the invention.

### DRAWINGS

FIG. 1. Effect on iron bioaccessibility with the combination of lutein and CPP (p<0.05). Horizontal bars indicate significant differences between compared conditions.

FIG. 2. Effect on iron bioaccessibility with the combination of lutein, CPP and short-chain fatty acids (p<0.05). Horizontal bars indicate significant differences between compared conditions.

FIG. 3. Effect on iron bioaccessibility with the combination of lutein, OPO and short-chain fatty acids (p<0.05). Horizontal bars indicate significant differences between compared conditions.

FIG. 4. Effect on calcium bioaccessibility with the combination of lutein, OPO and short-chain fatty acids (p<0.05). Horizontal bars indicate significant differences between compared conditions.

### DETAILED DESCRIPTION OF THE INVENTION

The invention relates to the non-therapeutic use of a composition comprising lutein for increasing mineral bioaccessibility in a human subject, wherein the composition further comprises at least one of:
a. calcium phosphopeptide, or
b. 1,3-dioleoyl-2-palmitoylglycerol.

The invention also relates to a composition comprising lutein for use in preventing or treating mineral deficiency and/or a condition associated therewith in a human subject, wherein the composition further comprises at least one of:
a. calcium phosphopeptide, or
b. 1,3-dioleoyl-2-palmitoylglycerol.

In some jurisdictions, this aspect may be defined as a method of preventing or treating mineral deficiency and/or a condition associated therewith in a human subject, the method comprising administering to the subject a composition comprising lutein and at least one of:
a. calcium phosphopeptide, or
b. 1,3-dioleoyl-2-palmitoylglycerol.

The invention may also be formulated as the use of lutein and at least one of:
a. calcium phosphopeptide, or
b. 1,3-dioleoyl-2-palmitoylglycerol in the manufacture of a composition for preventing or treating mineral deficiency and/or a condition associated therewith in a human subject.

The lutein is present in the composition in a therapeutically efficient amount.

Prevention, as used herein, refers to reduction of the risk of developing symptoms or conditions associated with mineral deficiency in a human subject, particularly a human subject at risk of developing such symptoms or conditions. The treatments encompassed by the present invention include alleviation of symptoms or attenuation of manifestation of the conditions associated with mineral deficiency.

As used herein, “mineral bioaccessibility” is defined as the amount of the ingested micronutrient (mineral) that is available for absorption in the gut after digestion. Preferably, increasing mineral bioaccessibility refers to increasing iron, zinc, magnesium and/or calcium bioaccessibility, more preferably iron and/or calcium bioaccessibility. “Mineral deficiency” refers to a reduced level of minerals essential to human health. An abnormally low mineral concentration is usually defined as a level that may impair a function dependent on that mineral.

Mineral deficiency, also known as micronutrient deficiency or “hidden hunger”, is a form of undernutrition that occurs when intake or absorption of minerals is too low to sustain good health and development in children and normal physical and mental function in adults. Causes include poor diet, gastrointestinal diseases altering the bioaccessibility of micronutrients, drugs interactions, or increased micronutrient needs, e.g. during pregnancy, lactation or growth.

In one embodiment, the prevention or treatment of mineral deficiency refers to zinc deficiency, iron deficiency or calcium deficiency, and/or a condition associated therewith in a human subject. Preferred conditions associated with mineral deficiency include iron malabsorption, anaemia, rachitis, impaired cognitive development (particularly delayed cognitive development), retarded growth, bone diseases (particularly osteoporosis), restless legs syndrome, impaired immune function, heart and lung problems, tiredness, postpartum depression, and the like. Preferably, the condition is selected from iron malabsorption, anaemia, cognitive impairment, osteoporosis, rachitis, growth retardation or impaired immune function.

Preferably, mineral deficiency is selected from calcium and iron. Preferably, the use in prevention or treatment of zinc, iron and/or calcium deficiency, preferably iron and/or calcium deficiency and associated conditions is directed to an infant, young child, child, lactating woman and/or an adult, preferably a female adult. More preferably, the human is an infant or infant child or female adult. More preferably the female adult is 45 years old or older, even more preferably 50 years old or older.

### Lutein

As used herein, lutein is in the form of free xanthophylls, xanthophyll esters or other chemical forms of lutein. Lutein may be obtained or isolated by any method recognized by those skilled in the art. For example, lutein may be obtained by extraction from marigolds or other xanthophylls-rich sources, chemical synthesis, fermentation or other biotechnology-derived and enriched xanthophyll sources. A suitable form of lutein useful in the present invention is available commercially as e.g. Floraglo® Lutein (powder with lutein at 1%), or commercially available lutein in an oily form. The herein defined amounts of lutein refer to free lutein (i.e., equivalent to 100% pure lutein).

In a preferred embodiment, the composition comprises lutein in an amount ≥60 μg per 100 g of composition, preferably 60-430 μg per 100 g of composition, more preferably 70-400 μg per 100 g of composition, even more preferably 70-350 μg per 100 g of composition, most preferably 80-350 μg per 100 g of composition. Preferably, the composition is a powder composition and the weight is expressed per 100 g of powder composition. Expressed differently, the composition comprises lutein in an amount of ≥5 μg/100 ml, preferably 5-65 μg/100 ml of the composition, preferably 7-60 μg/100 ml of the composition, more preferably 8-55 μg/100 ml of the composition, even more preferably 8.5-50 μg/100 ml of the composition. The amounts expressed per 100 ml of the composition refer to ready-to-drink nutritional composition in liquid form, for example, after reconstituting the powder composition in water.

According to one preferred embodiment, the composition is an infant formula. Preferably, the infant formula according to the invention comprises lutein in an amount of 60-430 μg per 100 g of composition, preferably, 70-400 μg per 100 g of composition, even more preferably 70-150 μg per 100 g of composition. Expressed differently, the infant formula comprises lutein in an amount of 5-65 μg/100 ml of the composition, preferably 6.5-60 μg/100 ml of the composition, more preferably 8-60 μg/100 ml of the composition, even more preferably 8-30 μg/100 ml of the composition. The amounts expressed per 100 ml of the infant formula refer to ready-to-drink infant formula in liquid form, for example, after reconstituting the powder in water.

According to another preferred embodiment, the composition is a follow-on formula. Preferably, the follow-on formula according to the invention comprises lutein in an amount of 60-430 μg per 100 g of powder composition, preferably, 120-390 μg per 100 g of powder composition, more preferably, 180-350 μg per 100 g of powder composition. Expressed differently, the follow-on formula comprises lutein in an amount of 5-65 μg/100 ml of the composition, preferably 15-55 μg/100 ml of the composition, more preferably 20-45 μg/100 ml of the composition. The amounts expressed per 100 ml of the follow-on formula refer to ready-to-drink follow-on formula in liquid form, for example, after reconstituting the powder composition in water.

According to yet another embodiment, the composition is a young child formula. Preferably, the young child formula according to the invention comprises lutein in an amount of 60-430 μg per 100 g of powder composition, preferably, 120-390 μg per 100 g of powder composition, more preferably, 180-350 μg per 100 g of powder composition. Expressed differently, the young child formula comprises lutein in an amount of 5-65 μg/100 ml of the composition, preferably 15-55 μg/100 ml of the composition, more preferably 20-45 μg/100 ml of the composition. The amounts expressed per 100 ml of the young child formula refer to ready-to-drink young child formula in liquid form, for example, after reconstituting the powder composition in water.

### Casein Phosphopeptides (CPP)

As used herein, casein phosphopeptides are defined as casein-derived peptides having at least one phosphoserine residue per peptide molecule. Casein phosphopeptides normally contain phosphoserine (SerP) residue, preferably at least 1 residue per 20 amino acid residues, more preferably at least 1 SerP residue per 10 amino acid residues or even at least 1 SerP per 7, and e.g. up to 3 SerP per 7 amino acid residues. In addition to or instead of SerP, other phosphorylated amino acids, such as phosphothreonine (ThrP) or phosphotyrosine (TyrP) may be present. Suitable casein phosphopeptides to be used in the invention can have phosphorus content between 0.6 and 1.5 wt %. Casein phosphopeptides can be prepared by enzymatic hydrolysis of casein or caseinate, especially whole casein, α-caseins, κ-casein or β-casein, for example using trypsin, pepsin, chymotrypsin, pancreatin or bacterial (Bacillus), fungal or plant endo- and/or exoproteases or mixtures thereof. Commercial sources of casein phosphopeptides include, but are not limited to, bovine casein phosphopeptides.

According to one embodiment, the composition comprises casein phosphopeptides (CPP). Preferably, the composition comprises CPP in an amount of 5-20 mg per 100 ml of the composition, preferably 6.0-16.0 mg per 100 ml of composition. The amounts expressed per ml of the composition refer to ready-to-drink nutritional composition in liquid form. Per 100 g of the composition, preferably a powder composition, CPP is present in an amount ranging from 25-159 mg, preferably 40-150 mg, more preferably 40-70 mg. Preferably, the weight ratio of lutein to CPP ranges from 1:150 to 1:1000, preferably from 1:200 to 1:900.

1,3-dioleoyl-2-palmitoyl plyceride (OPO)

Preferably, the composition comprises 1,3-Dioleoyl-2-palmitoyl glycerol (OPO). The triglyceride 1,3-dioleoyl-2-palmitoyl glyceride is known to be an important component of human milk fat.

As used herein, 1,3-dioleoyl-2-palmitoylglycerol refers to fats commercially available, for example, as Betapol® from Loders Croklaan B V, Wormerveer, The Netherlands such as Betapol® B-55.

According to preferred embodiments, the present invention relates to the combined use of lutein with 1,3-dioleoyl-2-palmitoylglycerol. Accordingly, the compositions, uses and methods of the invention preferably relate to a composition comprising lutein and 1,3-dioleoyl-2-palmitoylglycerol.

Preferably, the composition comprises 2-12 g OPO per 100 g composition, preferably 2.5-10 g OPO per 100 g composition, preferably a powder composition. Expressed differently, the composition comprises 0.27-1.62 g OPO per 100 ml of the composition, preferably 0.34-1.35 g OPO per 100 ml of the composition, preferably the reconstituted, ready-to-drink composition.

The inventors have surprisingly found that a combination of lutein, 1,3-dioleoyl-2-palmitoylglycerol and the fermentation products of non-digestible carbohydrates, such as short chain fatty acids, can synergistically improve mineral bioaccessibility. In particular, the inventors have surprisingly observed that calcium and iron bioaccessibility is synergistically improved.

Accordingly, in one aspect, the present invention relates to the non-therapeutic use of a composition comprising lutein for increasing mineral bioaccessibility in a human subject, wherein the composition further comprises 1,3-dioleoyl-2-palmitoylglycerol and at least one non-digestible carbohydrate. The invention thus further relates to a composition comprising lutein for use in preventing or treating mineral deficiency and/or a condition associated therewith in a human subject, wherein the composition further comprises 1,3-dioleoyl-2-palmitoylglycerol and at least one non-digestible carbohydrate.

### Non-Digestible Carbohydrates

According to a preferred embodiment, the composition according to the invention comprises non-digestible carbohydrates.

As used herein, the term “non-digestible carbohydrate” refers to oligosaccharides which are not digested in the intestine by the action of acids or digestive enzymes present in the human upper digestive tract, e.g. small intestine and stomach, but reach the distal portions of the intestines, such as the colon, intact where they are fermented by the human intestinal microbiota. For example, sucrose, lactose, maltose and maltodextrins are considered digestible saccharides.

Microbial metabolites of non-digestible carbohydrates include short chain fatty acids (SCFA), for example acetate, propionate, butyrate, lactate, among others. The inventors have surprisingly found that the presence of the fermentation products of non-digestible carbohydrates (SCFA's) further improves mineral bioaccessibility when used in combination with lutein and at least one of calcium phosphopeptide or 1,3-dioleoyl-2-palmitoylglycerol. Accordingly, in preferred embodiments, the composition comprises at least one non-digestible carbohydrate.

The composition according to the invention preferably comprises non-digestible carbohydrates selected from group comprising prebiotic oligosaccharides, human milk oligosaccharides, and combinations thereof.

Preferably, the composition comprises 80 mg to 4 g non-digestible carbohydrates per 100 ml, more preferably 150 mg to 2 g per 100 ml, even more preferably 300 mg to 1 g non-digestible carbohydrates per 100 ml. Based on dry weight, the composition preferably comprises non-digestible carbohydrates in an amount of 0.25 wt. % to 25 wt. % non-digestible carbohydrates, more preferably 0.5 wt. % to 10 wt. %, even more preferably 1.5 wt. % to 7.5 wt. %, based on total composition.

According to one preferred embodiment, the composition comprises calcium phosphopeptide and at least one non-digestible carbohydrate. Preferably, calcium, zinc and/or iron bioaccessibility is increased, more preferably iron bioaccessibility is increased. Therapeutic uses and methods for preventing or treating mineral deficiency and/or a condition associated therewith are herein encompassed.

According to another preferred embodiment, the composition comprises 1,3-dioleoyl-2-palmitoylglycerol and at least one non-digestible carbohydrate. Preferably, calcium, zinc and/or iron bioaccessibility is increased, more preferably iron and/or calcium bioaccessibility is increased.

Therapeutic uses and methods for preventing or treating mineral deficiency and/or a condition associated therewith are herein encompassed.

### Prebiotic Oligosaccharides

Prebiotic oligosaccharides are non-digestible oligosaccharides. Preferably, the nutritional composition according to the invention preferably comprises prebiotic oligosaccharides.

Preferred prebiotic oligosaccharides have a DP in the range of 2 to 250, more preferably 2 to 60, most preferably below 40. Advantageously and most preferred, the prebiotic oligosaccharides are water-soluble (according to the method disclosed in L. Prosky et al, J. Assoc. Anal. Chem 71: 1017-1023, 1988).

Suitable prebiotic oligosaccharides are at least one, more preferably at least two, preferably at least three selected from the group consisting of fructo-oligosaccharides, galacto-oligosaccharides, xylo-oligosaccharides, arabino-oligosaccharides, arabinogalacto-oligosaccharides, gluco-oligosaccharides, chito-oligosaccharides, glucomanno-oligosaccharides, galactomanno-oligosaccharides, mannan-oligosaccharides, and uronic acid oligosaccharides. The group of fructo-oligosaccharides includes inulins, the group of galacto-oligosaccharides includes transgalacto-oligosaccharides or beta-galacto-oligosaccharides, the group of gluco-oligosaccharides includes cyclodextrins, gentio- and nigero-oligosaccharides and non-digestible polydextrose, the group of galactomanno-oligosaccharides includes partially hydrolyzed guar gum, and the group of uronic acid oligosaccharides includes pectin degradation products (e.g. prepared from apple pectin, beet pectin and/or citrus pectin).

Preferably, the composition comprises at least one prebiotic oligosaccharide, more preferably at least two prebiotic oligosaccharide. Preferably, the composition comprises prebiotic oligosaccharides selected from the list comprising: fructo-oligosaccharides, galacto-oligosaccharides, or mixtures thereof. More preferably, the composition comprises fructo-oligosaccharides and galacto-oligosaccharides at a weight ratio between (20 to 2):1, more preferably (20 to 2):1, even more preferably (20 to 2):1, even more preferably (12 to 7):1. Most preferably the weight ratio is about 9:1.

The galacto-oligosaccharides preferably are beta-galacto-oligosaccharides. In a particularly preferred embodiment the present composition comprises beta-galacto-oligosaccharides ([galactose]n-glucose; wherein n is an integer ranging from 2 to 60, i.e. 2, 3, 4, 5, 6, . . . , 59, 60; preferably n is selected from 2, 3, 4, 5, 6, 7, 8, 9, and 10), wherein the galactose units are in majority linked together via a beta linkage. Beta-galacto-oligosaccharides are also referred to as trans-galacto-oligosaccharides (TOS). Beta-galacto-oligosaccharides are for example sold under the trademark Vivinal™ (Borculo Domo Ingredients, Netherlands). Another suitable source is Bi2Munno (Classado). Preferably the TOS comprises at least 80% beta-1,4 and beta-1,6 linkages based on total linkages, more preferably at least 90%.

Fructo-oligosaccharide is a prebiotic oligosaccharide comprising a chain of beta-linked fructose units with a DP or average DP of 2 to 250, more preferably 2 to 100, even more preferably 10 to 60. Fructo-oligosaccharide includes inulin, levan and/or a mixed type of polyfructan. An especially preferred fructo-oligosaccharide is inulin. Fructo-oligosaccharide suitable for use in the compositions is also commercially available, e.g. Raftiline® HP (Orafti). Preferably the fructo-oligosaccharide has an average DP above 20.

In one preferred embodiment, the composition according to the invention comprises prebiotic oligosaccharides only, i.e., without human milk oligosaccharides.

Preferably, the composition comprises 80 mg to 4 g prebiotic oligosaccharides per 100 ml, more preferably 150 mg to 2 g per 100 ml, even more preferably 300 mg to 1 g prebiotic oligosaccharides per 100 ml. Based on dry weight, the composition preferably comprises prebiotic oligosaccharides in an amount of 0.25 wt. % to 25 wt. % prebiotic oligosaccharides, more preferably 0.5 wt. % to 10 wt. %, even more preferably 1.5 wt. % to 7.5 wt. %, based on total composition. Expressed per 100 g of the composition, prebiotics oligosaccharides are present in an amount of at least 3 g per 100 g composition, more preferably 3.5-8 g per 100 g composition. Typically, the composition is in powder form, for instance, an infant formula, follow-on formula and/or young child formula. More preferably, the prebiotic oligosaccharides are selected from galacto-oligosaccharides, fructo-oligosaccharides or a combination thereof.

### Human Milk Oligosaccharides

According to one embodiment, the nutritional composition according to the invention comprises human milk oligosaccharides. “Human milk oligosaccharides” (HMOs) are present in human milk and are non-digestible carbohydrates built from the following monomers: D-glucose, D-galactose, N-acetylglucosamine, L-fucose and sialic acid (N-acetylneuraminic acid).

Preferably, the composition of the invention comprises human milk oligosaccharides selected from the group comprising, but not limited to, sialyloligosaccharides, such as 3-sialyllactose (3-SL), 6-sialyllactose (6-SL), lactosialyltetrasaccharide a,b,c (LST), disialyllactoNtetraose (DSLNT), sialyl-lactoNhexaose (S-LNH), DS-LNH, and fucooligosaccharide, such as (un)sulphated fucoidan oligosaccharide, 2′-fucosyllactose (2′-FL), 3-fucosyllactose (3-FL), difucosyllactose, lacto-N-fucopenatose, (LNFP) I, II, III, IV Lacto-N-neofucopenaose (LNnFP), Lacto-N-difucosyl-hexaose (LNDH), and mixtures thereof.

In one preferred embodiment, the composition according to the invention comprises human milk oligosaccharides only, i.e., without prebiotic oligosaccharides.

In one preferred embodiment, the composition according to the invention comprises human milk oligosaccharides only, i.e., without prebiotic oligosaccharides. Based on dry weight, the present nutritional composition preferably comprises 0.038 wt. % to 12 wt. % HMOs, preferably 0.075 wt. % to 9 wt. % HMOs, more preferably 0.15 wt. % to 6 wt. % HMOs, even more preferably 0.3 wt. % to 2.5 wt. % HMOs. Expressed differently, the composition comprises human milk oligosaccharides in an amount of 0.5 mg to 5 g per 100 ml of the composition, preferably 1.0 mg to 4.5 g per 100 ml of the composition, more preferably 0.5 g to 4.0 g per 100 ml of the composition, even more preferably 1.0 g to 3.5 g per 100 ml of the composition, most preferably 1.5 g to 3.0 g/100 ml of the composition. The amounts expressed per ml of the composition refer to ready-to-drink nutritional composition in liquid form. Based on energy, the present nutritional composition preferably comprises 0.008 to 2.5 g HMOs per 100 kcal, preferably 0.015 to 2.5 g HMOs per 100 kcal, more preferably 0.03 to 1.0 g HMOs per 100 kcal, even more preferably 0.06 to 0.5 g HMOs per 100 kcal. A too high amount will result in an increase the risk of osmotic diarrhea, which will counteract the beneficial effects of the mix.

Preferably, human milk oligosaccharides are selected from 2′-fucosyllactose (2′-FL), 3-fucosyllactose (3-FL), 3-sialyllactose (3-SL), 6-sialyllactose (6-SL), lacto-N-tetrose (LNT), lacto-N-neotetrose (LNnT), or combinations thereof. More preferably, the composition comprises 2′-FL.

2′-FL, preferably α-L-Fuc-(1→2)-β-D-Gal-(1→4)-D-Glc, is commercially available for instance from Sigma-Aldrich. Alternatively, it can be isolated from human milk, for example as described in Andersson & Donald, 1981, J Chromatogr. 211:170-1744, or produced by genetically modified micro-organisms, for example as described in Albermann et al, 2001, Carbohydrate Res. 334:97-103.

The nutritional composition of the present invention preferably comprises at least one human milk oligosaccharide selected from the group consisting of 2′-FL, 3-FL, 3′-SL and 6′-SL.

In a preferred embodiment, a nutritional composition according to the invention comprises at least 0.005 g of the sum of 2′-FL, 3-FL, 3′-SL and 6′-SL per 100 ml, more preferably at least 0.01 g, more preferably at least 0.02 g, even more preferably at least 0.04 g of the sum of 2′-FL, 3-FL, 3′-SL and 6′-SL per 100 ml. Based on dry weight, the present nutritional composition preferably comprises at least 0.038 wt. % of the sum of 5 2′-FL, 3-FL, 3′-SL and 6′-SL, more preferably at least 0.075 wt. %, more preferably at least 0.15 wt. % of the sum of 2′-FL, 3-FL, 3′-SL and 6′-SL, even more preferably at least 0.3 wt. %. Based on energy, the present nutritional composition preferably comprises at least 0.008 g of the sum of 2′-FL, 3-FL, 3′-SL and 6′-SL per 100 kcal, more preferably at least 0.015 g per 100 kcal, more preferably at least 0.03 g per 100 kcal, even more preferably at least 0.06 per 100 kcal.

Preferably the nutritional composition according to the invention comprises as a HMOS essentially 2′-FL, that means at least 95 wt. % of the HMOS consists of 2′-FL. Preferably, a nutritional composition according to the invention comprises 0.01 g to 1 g 2′-FL per 100 ml, more preferably 0.02 g to 0.5 g, even more preferably 0.04 g to 0.2 g 2′-FL per 100 ml. Based on dry weight, the present nutritional composition preferably comprises 0.075 wt. % to 8 wt. % 2′-FL, more preferably 0.15 wt. % to 4 wt. % 2′-FL, even more preferably 0.3 wt. % to 1.5 wt. % 2′-FL. Based on energy, the present nutritional composition preferably comprises 0.015 to 1.5 g 2′-FL per 100 kcal, more preferably 0.03 to 0.75 g 2′-FL per 100 kcal, even more preferably 0.06 to 0.4 g 2′-FL per 100 kcal.

In yet another preferred embodiment, a mixture of prebiotic oligosaccharides and human milk oligosaccharides is present. Preferably, the composition comprises at least two different non-digestible carbohydrates wherein at least two non-digestible carbohydrates are selected from either the group of prebiotics oligosaccharides or from the group of human milk oligosaccharides.

According to another embodiment, the nutritional composition comprises non-digestible carbohydrates, preferably at least two different non-digestible carbohydrates, more preferably, two different sources of non-digestible carbohydrates. Preferably, the at least two different non-digestible carbohydrates include a non-digestible carbohydrate selected from the group of prebiotics oligosaccharides and a non-digestible carbohydrate selected from the group of human milk oligosaccharides. More preferably, the composition comprises galacto-oligosaccharide and fructo-oligosaccharide in combination with 2′-FL and/or LNT, preferably 2′-FL.

Preferably, the weight ratio of human milk oligosaccharides (for instance FL, preferably 2′-FL) to prebiotic oligosaccharide (preferably, galacto-oligosaccharide) is from 5 to 0.05, more preferably 5 to 0.1, more preferably from 2 to 0.1. Preferably the weight ratio human milk oligosaccharides (for instance FL, preferably 2′-FL) to prebiotic oligosaccharide (preferably, fructo-oligosaccharide, more preferably inulin) is from 10 to 0.05, more preferably 10 to 0.1, more preferably from 2 to 0.5.

### Nutritional Compositions and Other Components

The present composition is preferably enterally administered, more preferably orally. The composition of the present invention includes dry food, preferably a powder, which is accompanied with instructions as to admix said dry food mixture with a suitable liquid, preferably with water.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid.

The present composition preferably comprises a lipid component, protein component, carbohydrate component and combinations thereof.

Preferably, the nutritional composition according to the invention is a nutritionally complete composition, that is, the composition comprises lipids, carbohydrates, and proteins.

According to a preferred embodiment, the human is an infant or young child. Preferably, the infant or young child is between 0-60 months of age, more preferably 0-36 months of age, even more preferably 6-24 months of age, most preferably 6-18 months of age.

Accordingly, preferred nutritional compositions for infants or young children include infant formula, follow-on formula, young child formula/growing-up milk, milk fortifiers, nutritional supplements, etc. Preferably, the nutritional composition is an infant formula, follow-on formula, or young child formula/growing-up milk. Preferably, the compositions are in the form of powders for reconstitution in a liquid prior to consumption. The present composition can be advantageously applied as a complete nutrition for infants. The compositions are synthetic compositions, i.e., are not or does not comprise human breast milk.

In the present invention, infant formula refers to nutritional compositions, artificially made, intended for infants of 0 to about 4 to 6 months of age and are intended as a substitute for human milk. Typically, infant formulae are suitable to be used as sole source of nutrition. Such infant formulae are also known as starter formula. Follow-on formula for infants starting with at 4 to 6 months of life to 12 months of life are intended to be supplementary feedings for infants that start weaning on other foods. Infant formulae and follow-on formulae are subject to strict regulations, for example for the EU regulations no. 609/2013 and no. 2016/127. In the present context, young child formula refers to nutritional compositions, artificially made, intended for infants of 12 months to 36 months, which are intended to be supplementary feedings for infants. In the context of the present invention, young child formula can also be named growing-up milk.

Nutritional compositions for children are preferably for children between 3 to 12 years of age, preferably 2 to 10 years of age, more preferably 3 to 6 years of age. Non-limiting examples of compositions for children include nutritional supplements, e.g. powders to be dispersed in a liquid such as water, milk or yoghurt, or ready-to-drink beverages.

The nutritional composition is preferably an infant formula or a follow-on formula or young child formula.

In one embodiment, the nutritional composition is preferably an infant formula or follow-on formula or young child formula and preferably comprises 3 to 7 g lipid/100 kcal, preferably 4 to 6 g lipid/100 kcal, more preferably 4.5 to 5.5 g lipid/100 kcal, preferably comprises 1.7 to 3.5 g protein/100 kcal, more preferably 1.8 to 3.0 g protein/100 kcal, more preferably 1.8 to 2.5 g protein/100 kcal and preferably comprises 5 to 20 g digestible carbohydrate/100 kcal, preferably 6 to 16 g digestible carbohydrate/100 kcal, more preferably 10 to 15 g digestible carbohydrate/100 kcal.

The nutritional composition preferably has an energy density of 60 kcal to 75 kcal/100 ml, more preferably 60 to 70 kcal/100 ml, when in a ready-to-drink form.

Preferably, the composition comprises 5-15 g proteins per 100 g composition, more preferably 8-14 g proteins per 100 g composition. Normally the composition is a powder, therefore per 100 g powder composition. Expressed differently, the composition comprises 0.8-2.5 g proteins per 100 ml, more preferably 1.0-2.0 g proteins per 100 ml of the ready-to-drink composition. The protein is intact, partially or fully hydrolyzed.

Lipids are preferably present in an amount between 15-35 g per 100 g composition, preferably 20-30 g per 100 g composition. Expressed differently, the composition comprises 2.5-5 g lipids per 100 ml of the ready-to-drink composition, preferably 3.0-4.5 g lipids per 100 ml of the ready-to-drink composition.

Preferably, lipids include linoleic acid and alpha-linolenic acid. Preferably, the composition comprises 1.5-6 g linoleic acid per 100 g composition, preferably a powder composition. In another preferred embodiment, the composition comprises 150-550 mg alpha-linoleic acid per 100 g composition, preferably a powder composition.

Preferably the composition comprises DHA and/or ARA. Preferably, the composition comprises 65-150 mg DHA per 100 g composition, preferably a powder composition. Also preferably is a composition comprising from 100-200 mg ARA per 100 g composition, preferably a powder composition.

Preferably, the composition comprises digestible carbohydrates. Preferred digestible carbohydrate sources are lactose, glucose, sucrose, fructose, galactose, maltose, starch and maltodextrin. Preferably, the composition comprises at least 40 g digestible carbohydrates per 100 g of composition, preferably powder composition, more preferably 45-70 g digestible carbohydrates per 100 g composition. Per 100 ml, the composition comprises 5-9 g digestible carbohydrates, preferably 6-8 g digestible carbohydrates. The amounts expressed per 100 ml of the composition refer to ready-to-drink nutritional composition in liquid form, for example, after reconstituting the powder composition in water.

Lactose is the main digestible carbohydrate present in human milk. The nutritional composition preferably comprises lactose. The nutritional composition preferably comprises digestible carbohydrate, wherein at least 35 wt. %, more preferably at least 50 wt. %, more preferably at least 75 wt. %, even more preferably at least 90 wt. %, most preferably at least 95 wt. % of the digestible carbohydrate is lactose. Based on dry weight the nutritional composition preferably comprises at least 25 wt. % lactose, preferably at least 40 wt. % lactose.

In one embodiment, the composition is selected from infant formula, follow on formula, growing up milk, human milk fortifier, supplements, baby food, weaning products, or the like. In another embodiment, the human subject is an adult, preferably an aging adult (e.g., an adult above 45 years old), more preferably a female adult, even more preferably above 45 years of age, most preferably above 55 years of age.

Non-limiting examples of compositions include fortified foods, supplements, nutraceuticals, capsules, powders, juices, milk powders, morning or evening supplements, and the like.

According to a preferred embodiment, the composition comprises minerals. Preferably, the minerals are selected from the group comprising iron, zinc, magnesium, calcium, or combinations thereof.

Preferably, the composition comprises an iron source. Iron is present in amount ranging between 1.0 to 10 mg/100 g of the total composition, preferably 1.5 to 9.0 mg/100 g of the total composition, even more preferably 2.0 to 8.5 mg/100 g of the total composition. Per 100 ml of the reconstituted composition, iron is present in an amount ranging between 0.2 to 1.5 mg, preferably 0.25 to 1.25 mg, even more preferably 0.3 to 1.0 mg iron per 100 ml of the composition.

Preferably, the composition comprises an calcium source. Calcium is present in amount ranging between 200 to 1000 mg/100 g of the total composition, preferably 250 to 850 mg/100 g of the total composition, even more preferably 280 to 750 mg/100 g of the total composition. Per 100 ml of the reconstituted composition, iron is present in an amount ranging between 25 to 100 mg, preferably 30 to 90 mg, even more preferably 35 to 85 mg iron per 100 ml of the composition.

Preferably, the composition comprises an iron source such as ferrous sulphate, ferrous fumarate, ferrous gluconate, iron dextran, sodium ferrous citrate, iron tartrate, etc. Iron is present in an amount ranging between 1.0 to 10 mg/100 g of the total composition, preferably 1.5 to 9.5 mg/100 g of the total composition, even more preferably 2.0 to 9.0 mg/100 g of the total composition. Per 100 ml of the reconstituted composition, iron is present in an amount ranging between 0.13 to 1.35 mg, preferably 0.20 to 1.29 mg, even more preferably 0.27 to 1.215 mg iron per 100 ml of the composition.

Preferably, the composition comprises a zinc source such as zinc sulfate, zinc lactate, zinc gluconate zinc stearate etc. Zinc is present in amount ranging between 1.0 to 10 mg/100 g of the total composition, preferably 1.5 to 9.5 mg/100 g of the total composition, even more preferably 2.0 to 9.0 mg/100 g of the total composition. Per 100 ml of the reconstituted composition, iron is present in an amount ranging between 0.13 to 1.35 mg, preferably 0.20 to 1.29 mg, even more preferably 0.27 to 1.215 mg zinc per 100 ml of the composition.

Preferably, the composition comprises a magnesium source such as magnesium chloride, magnesium citrate, magnesium gluconate, magnesium malate, magnesium sulfate, magnesium-L-hreonate etc. Magnesium is present in amount ranging between 10.0 to 110 mg/100 g of the total composition, preferably 15 to 105 mg/100 g of the total composition, even more preferably 20 to 100 mg/100 g of the total composition. Per 100 ml of the reconstituted composition, magnesium is present in an amount ranging between 1.35 to 14.85 mg, preferably 2.025 to 14.175 mg, even more preferably 2.7 to 13.5 mg magnesium per 100 ml of the composition.

Preferably, the composition comprises an calcium source such as tricalcium phosphate, calcium hydrogen phosphate, calcium carbonate, calcium d-pantothenate, calcium hydroxide, calcium lactate, calcium gluconate, milk calcium, active calcium, organic calcium, calcium alginate etc. Calcium is present in amount ranging between 200 to 1100 mg/100 g of the total composition, preferably 250 to 1000 mg/100 g of the total composition, even more preferably 280 to 850 mg/100 g of the total composition. Per 100 ml of the reconstituted composition, iron is present in an amount ranging between 27 to 148.5 mg, preferably 33.75 to 135 mg, even more preferably 37.8 to 114.75 mg iron per 100 ml of the composition.

In a further aspect, the invention relates to a composition comprising carbohydrates, lipids, proteins and an energy content of at least 400 kcal per 100 g of the composition, wherein the composition comprises:
a. 60-430 μg lutein per 100 g of composition,
b. 1.0 to 10 mg iron per 100 g of the total composition, wherein the composition further comprises at least one of:
c. 25-80 mg casein phosphopeptides per 100 g of composition or
d. 2-10 g 1,3-dioleoyl-2-palmitoylglycerol per 100 g composition.

Preferably, the composition comprises non digestible carbohydrates, preferably 3.5-8 g non-digestible carbohydrates per 100 g of the composition.

More preferably, the composition comprises 45-70 g digestible carbohydrates, preferably lactose.

All other optional ingredients described above for the uses and methods of the invention apply for the composition mutatis mutandis.

The invention is described by means of non-limiting examples below.

### EXAMPLES

In vitro simulated gastrointestinal digestion was used for assessing mineral bioaccessibility in presence of the tested compounds.

### Materials

Mineral bioaccessibility buffer (MBB) was prepared (NaCl; 6.6 mg/mL, MgSO4; 0.12 mg/mL, Glucose; 0.9 mg/mL, L-Ascorbic acid; 0.09 mg/mL, HEPES; 8.6 mg/mL at pH 6.5) with Calcium, Iron and Zinc (tricalcium phosphate; 1314.6 μg/mL, Calcium D-pantothenate; 28.5 μg/mL, Calcium Carbonate; 13.0 μg/mL, Iron Sulphate heptahydrate; 3.8 μg/mL, Iron Chloride; 12.5 μg/mL, Zinc Sulphate heptahydrate; 17.4 μg/mL, Zinc Chloride; 2.2 μg/mL) to achieve a total mineral content (Calcium; 520 μg/mL, Iron; 5.0 μg/mL, Zinc; 5.0 μg/mL), as well as soluble mineral content similar to that of infant formula.

Tested compound solutions were prepared in concentrated stock according to their solubility; casein phosphopeptides (CPP) 100× in 50 mM NaOH, lutein 241× in 310 mM taurocholate/64.5 mM phosphatidylcholine, 2-palmitoylglycerol (2-PG, a proxy for the lipolytic derivative of 1,3-dioleoyl-2-palmitoylglycerol) 20× in EtOH, palmitic acid 20× (PA) in DMSO, short chain fatty acids (SCFA, 75 mol % acetic acid, 20 mol % propionic acid, 5 mol % butyric acid) 1000× in DMEM.

Subsequently, CPP, lutein or 2-PG/PA were added to 35 mL of MBB reaching final concentrations of 6.16 mg/100 mL, 41.5 μg/100 mL and 5 mM respectively. SCFA were added to the MBB at the start of the colonic phase of the mineral bioaccessibility model to a final concentration of 4 mM.

### Method

The transit of CCP, Lutein, 2-PA and non-digestible Gos/Fos through the gastro-intestinal tract was mimicked using a three-phase dialysis model that was adapted from literature [Venema, 2020]. For the gastric phase, 35 mL MBB in a 50 mL vial was placed in a water bath at 37° C., acidified to pH 4.0 and incubated under constant agitation for 1 hour. Continuing with the intestinal phase, the pH was raised to 6.5 and 20 mL was transferred to a dialysis tube (SIGMA, D0530, MWCO 12.4 kDa) with trypsin added at 0.09 mg/mL, the tube was clipped at both ends. The resulting dialysis bag was placed in a volumetric cylinder containing 500 mL succinic buffer (0.05 M succinic acid, pH 6.5) and incubated for 3h hours at 37° C. with magnetic stirring at 200 rpm. Lastly, dialysis continued for an additional hour in the colonic phase. The effect of colonic fermentation of non-digestible Gos/Fos 9:1 on mineral bioaccessibility was simulated by adding the resulting SCFA to the dialysis bag. Additionally, the MBB and succinic buffer were acidified to pH 5.5, in accordance with findings from a clinical study where infants that were fed Infant formula with Gos/Fos were found to have a more acidic fecal pH [Béghin, 2021]. Finally, the dialysis bag was removed, and the dialysate was sampled and stored at −20° C. An arm consisting of MBB without mineral bioaccessibility enhancers was included as reference. Calcium, Iron and Zinc in the dialysate were analyzed with ICP-OES spectrometry.

### Data Analysis

The average (n=3) total amount of Calcium, Iron and Zinc measured in the dialysate was considered bioaccessible. The total bioaccessible Calcium, Iron and Zinc of each treatment condition was expressed relative to that of the reference arm. ANOVA followed by Dunnett post-hoc analyses was used to compare the reference arm versus the other study arms containing the potential mineral bioaccessibility enhancers. To determine statistical significant differences between the potential mineral bioaccessibility enhancers and combinations thereof ANOVA was followed by LSD post-hoc analyses. Differences were considered statistically significant at p<0.05.

### Results

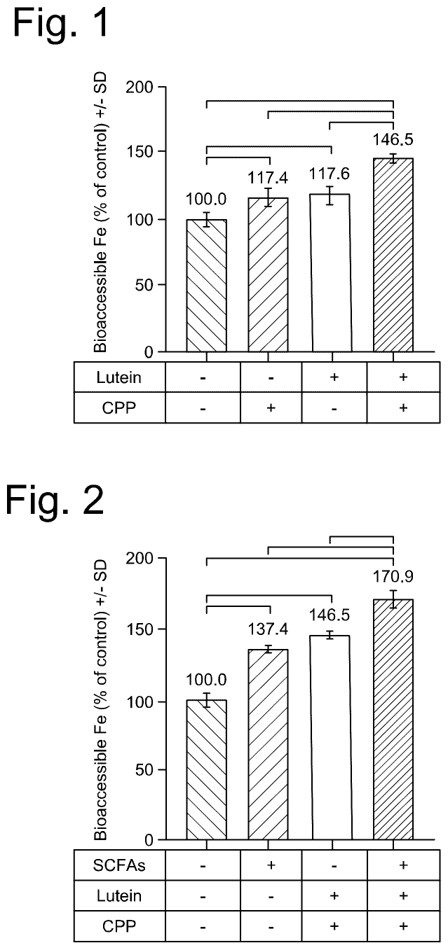

The results are shown in the Figures. The combination of lutein with casein phosphopeptide provides a synergistic effect on iron bioaccessibility (p<0.05) as compared to control or with the individual components (horizontal bars indicate significant differences between compared conditions)—FIG. 1. It was further observed that the addition of short-chain fatty acids to the combination of lutein and casein phosphopeptide significantly increased iron bioaccessibility when compared to the combination without the short-chain fatty acids, control and short-chain fatty acids alone—FIG. 2.

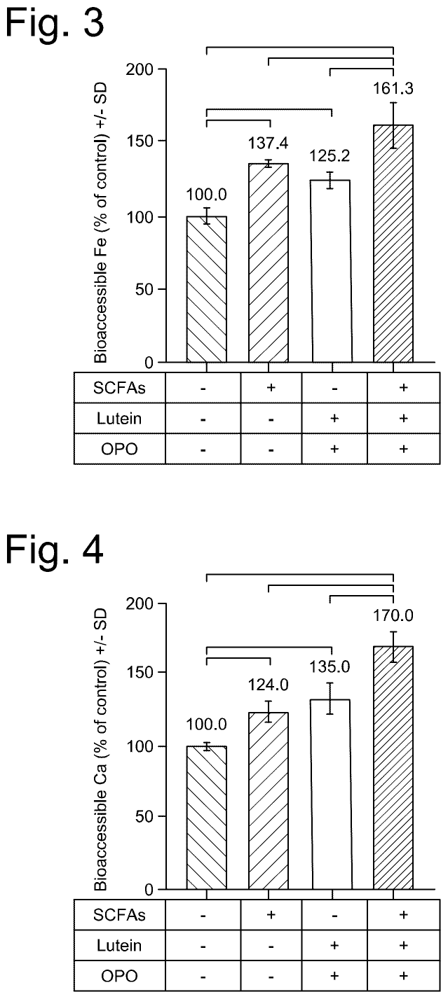

FIGS. 3 and 4 show the synergistic effect of the combination of lutein, OPO and short-chain fatty acids on iron (FIG. 3) and calcium (FIG. 4) bioaccessibility.

### REFERENCES

[Venema, 2020] Koen Venema, Jessica, Verhoeven, Sanne Verbruggen. Calcium and phosphorus bioaccessibility from different amino acid-based medical nutrition formulas for infants and children under in vitro digestive conditions, Clinical Nutrition Experimental, 2020
[Béghin, 2021] Laurent Béghin, Sebastian Tims, Mieke Roelofs. Fermented infant formula (with Bifidobacterium breve C50 and Streptococcus thermophilus 065) with prebiotic oligosaccharides is safe and modulates the gut microbiota towards a microbiota closer to that of breastfed infants, Clinical Nutrition, 2021

1.-18. (canceled)

19. A method for increasing mineral bioaccessibility in a human subject, the method comprising administering a composition comprising lutein and further comprising at least one of:
a. calcium phosphopeptide, or
b. 1,3-dioleoyl-2-palmitoylglycerol.

20. The method according to claim 19, wherein the composition comprises ≥60 μg lutein per 100 g of composition, preferably 60-430 μg per 100 g of composition.

21. The method according to claim 19, wherein the mineral is selected from the group comprising iron, calcium, zinc, magnesium, or combinations thereof.

22. The method according to claim 19, wherein the composition comprises casein phosphopeptides.

23. The method according to claim 19, wherein the composition comprises 25-159 mg casein phosphopeptides per 100 g of composition.

24. The method according to claim 19, wherein the composition comprises 1,3-dioleoyl-2-palmitoylglycerol.

25. The method according to claim 19, wherein the composition comprises 2-12 g per 100 g composition.

26. The method according to claim 19, wherein the composition comprises non-digestible carbohydrates, preferably selected from prebiotic oligosaccharides, human milk oligosaccharides, or combinations thereof.

27. The method according to claim 26, wherein the composition comprises prebiotic oligosaccharides, preferably selected from fructo-oligosaccharides (FOS), galacto-oligosaccharides (GOS), or mixtures thereof.

28. The method according to claim 26, wherein the composition comprises human milk oligosaccharides.

29. The method according to claim 28, wherein the oligosaccharides is selected from 2′-fucosyllactose (2′-FL), 3-fucosyllactose (3-FL), 3-sialyllactose (3-SL), 6-sialyllactose (6-SL), lacto-N-tetrose (LNT), lacto-N-neotetrose (LNnT), or combinations thereof.

30. The method according to claim 28, wherein the human milk oligosaccharide is 2′-fucosyllactose (2′-FL).

31. The method according to claim 19, wherein the composition comprises 1.0 to 10 mg iron per 100 g of the total composition.

32. The method according to claim 19, wherein the human subject is an infant or young child.

33. The method according to claim 19, wherein the human subject is an adult.

34. A method for the prevention or treatment of a mineral deficiency and/or a condition associated therewith in a human subject, wherein the method comprises administering a composition comprising lutein, and wherein the composition further comprises at least one of:
a. calcium phosphopeptide, or
b. 1,3-dioleoyl-2-palmitoylglycerol.

35. The method according to claim 34, wherein the condition is iron malabsorption, anaemia, rachitis, impaired cognitive development, retarded growth, bone disease, restless legs syndrome, impaired immune function, heart and lung problems, tiredness, postpartum depression, and combinations thereof.

36. A composition comprising carbohydrates, lipids, proteins and an energy content of at least 400 kcal per 100 g of the composition, wherein the composition comprises:
c. 60-430 μg lutein per 100 g of composition,
d. 1.0 to 10 mg iron per 100 g of the total composition,
wherein the composition further comprises at least one of:
e. 25-80 mg casein phosphopeptides per 100 g of composition or
f. 2-10 g, preferably 2-9 g 1,3-dioleoyl-2-palmitoylglycerol per 100 g composition.
